(19) United States
(12) Patent Application Publication
(10) Pub. No.: US 2024/0108360 A1
(43) Pub. Date: Apr. 4, 2024

(54) SURGICAL TOOL AND CORRESPONDING PRODUCTION METHOD

(71) Applicant: HPF S.R.L., Fagagna (IT)

(72) Inventors: Gabriele LUALDI, Fagagna (IT); Pablo DE AGOSTINI, Moimacco (IT)

(21) Appl. No.: 18/280,139

(22) PCT Filed: Feb. 25, 2022
(86) PCT No.: PCT/IT2022/050035

§ 371 Date: Sep. 1, 2023

(30) Foreign Application Priority Data
Mar. 2, 2021 (IT) 102021000004808

(51) Int. Cl. A61B 17/16

(52) U.S. Cl. CPC A61B 17/164; A61B 2017/00526

(57) ABSTRACT

A surgical tool for preparing a bone canal including a cutting unit configured to remove and/or compact bone tissues, which in turn a plurality of autonomous cutting members independent from each other and disposed each one in contact with the adjacent one.

### FIELD OF THE INVENTION

The present invention concerns a surgical tool, in particular for prosthetic surgery, suitable for the removal and/or compaction of bone tissues in the context of surgical interventions for the implantation of orthopedic prostheses, as well as the method to produce it. The surgical tool of the present invention is applied in the preparation of any bone canal for the implantation of a shaped stem of a prosthesis such as, for example, but without limitations in general, a femoral prosthesis, a prosthesis of the humerus, tibia or shoulder, and others.

### BACKGROUND OF THE INVENTION

Surgical tools are known, such as for example broaches or rasps, used for the preparation of a bone canal or seating, for the implantation of orthopedic prostheses, for example the stem of a femoral prosthesis, a prosthesis of the humerus, the tibia, the shoulder and others.

This type of surgical tool normally has an elongated shape, tapered and mating with that of the stem of the prosthesis to be implanted and, generally, they are forcibly inserted into a hole made in a portion of bone prepared in advance, so as to widen it and give it a desired shape, for example with a square or rectangular cross section.

Known surgical tools comprise at a first end a connector for a tool holder, at a second end a penetration tip and a cutting surface disposed between the two ends.

The shape of the cutting surface is different depending on the use of the tool.

In particular, the cutting surface of broaches is conformed as steps with edges that can be either sharp, or rounded or beveled, to act adequately on the bone tissue to remove it or to compact it.

On the contrary, the cutting surface of rasps comprises a plurality of small cutting elements conformed as punctiform protuberances that protrude from the lateral surface of the tool and the main function of which is to remove the bone tissue.

These known surgical tools are generally made in a single body, that is, without a break in continuity, substantially in two alternative ways: a) by forging, or b) by machining with removal of chip, usually using numerically controlled machine tools.

Furthermore, in both of these alternative methods, a grinding of the semi-finished surgical tool can follow, in order to finish the cutting surface.

One disadvantage of known surgical tools is that they have rather high production costs.

In fact, in the case of forging, it is necessary to prepare a hammer and the corresponding dies, which require high initial investments, while machining with chip removal, in addition to a considerable initial investment for the machine tool, requires long machining times; therefore, in both cases, there is a high production cost for each surgical tool made.

In addition, another disadvantage is that both of these production methods are not very flexible in terms of customizing the surgical tool which instead, in practice, requires a great deal of customization.

Document US-A-2010/262146 describes a reaming or milling instrument for prosthetic surgery.

Document U.S. Pat. No. 5,006,121 describes a broach for the femur or other bones for preparing them for the purpose of implanting a prosthesis, for example a hip prosthesis.

Document U.S. Pat. No. 4,412,733 discloses a reamer or miller for acetabular prostheses.

Document BE-A-1.015.812 describes a rasp for prosthetic surgery, in particular for implanting a prosthesis in the femur.

There is therefore a need to make a surgical tool, and to perfect corresponding new and original manufacturing methods, which can overcome at least one, better if all, of the disadvantages of the state of the art.

In particular, one purpose of the present invention is to provide a surgical tool which is economical to make, and which can be easily customized according to the needs of the end user, who is normally a surgeon, or a professional in the field of prosthetics.

Another purpose of the present invention is to perfect a method to produce a surgical tool which is quick, economical and flexible in terms of customization of the surgical tool.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes, a surgical tool for the preparation of a bone canal for the implant of the stem of a prosthesis comprises at least one cutting unit configured to remove and/or compact bone tissues.

According to one aspect of the present invention, the cutting unit comprises a plurality of autonomous cutting members independent from each other, disposed each one in contact with the adjacent one according to a determinate sequence along a common longitudinal axis.

According to another aspect of the present invention, the surgical tool also comprises a first element associated with the cutting unit and configured to be mechanically connected to an external tool holder.

According to another aspect of the present invention, each of the cutting members also comprises a central cavity substantially coaxial to the longitudinal axis and the plurality of central cavities of the cutting members disposed in sequence defines a central channel.

According to another aspect of the present invention, the surgical tool also comprises a central support member inserted inside the central channel and configured to give structural rigidity to the cutting unit.

According to another aspect of the present invention, the first element is disposed at a first end of the central support member and the surgical tool also comprises a second element disposed at a second end of the central support member, opposite the first end.

According to another aspect of the present invention, at least one of either the first element or the second element is made in a single body with the central support member.

In accordance with another aspect of the present invention, a method to produce a surgical tool comprises at least the following steps:
producing, or having available, a plurality of cutting members, each one distinct and separate from the other;
disposing the cutting members each one in contact with another adjacent one according to a determinate sequence along a common longitudinal axis in order to make up a cutting unit.

According to one aspect of the present invention, each of the cutting members is made with a central cavity and the plurality of central cavities defines a central channel of the cutting unit.

In these cases, the method of the present invention also comprises the following steps:
producing, or having available, a central support member mating in shape with the central channel;
inserting each of the cutting members on the central support member.

### BRIEF DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a three-dimensional view of a surgical tool according to the present invention in accordance with a first embodiment;

FIG. 2 is a three-dimensional exploded view of the surgical tool according to FIG. 1;

FIG. 3 is a three-dimensional view of a surgical tool according to the present invention in accordance with a second embodiment;

FIG. 4 is an exploded section view of the cutting unit of the surgical tool of FIG. 1;

FIG. 5 is a section view of the cutting unit of the surgical tool of FIG. 1;

FIGS. from 6 to 8 are section views of different embodiments of the surgical tool according to the present invention;

FIG. 9 is a lateral view of the surgical tool of FIG. 1;

FIGS. from 10 to 13 are schematic views of different embodiments of the cutting members of the surgical tool according to the present invention.

We must clarify that in the present description and in the claims the term vertical, with its declinations, has the sole function of better illustrating the present invention with reference to the drawings and must not be in any way used to limit the scope of the present invention, or the field of protection defined by the attached claims. For example, by the term vertical we mean an axis or a plane that can be either perpendicular to the line of the horizon, or inclined, even by several degrees, for example up to 20°, with respect to the latter.

Furthermore, the people of skill in the art will recognize that certain sizes or characteristics in the drawings may have been enlarged, deformed, or shown in an unconventional or non-proportional way in order to provide a version of the present invention that is easier to understand. When sizes and/or values are specified in the following description, the sizes and/or values are provided for illustrative purposes only and must not be construed as limiting the scope of protection of the present invention, unless such sizes and/or values are present in the attached claims.

The phraseology and terminology used here are also solely for the purposes of providing non-limiting examples. Furthermore, all the intervals reported here shall be understood to include the extremes, including those that report an interval between two values, unless otherwise indicated.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

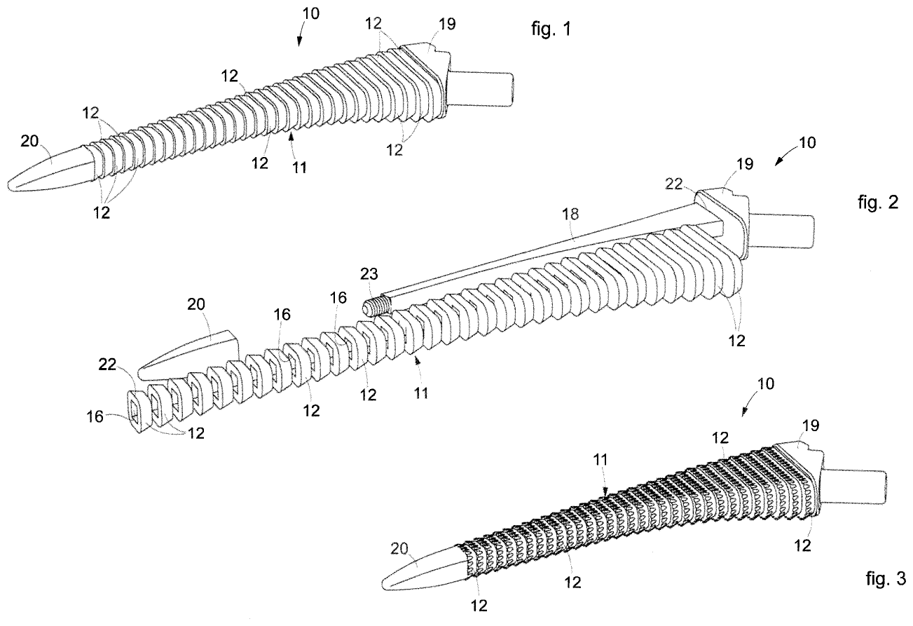

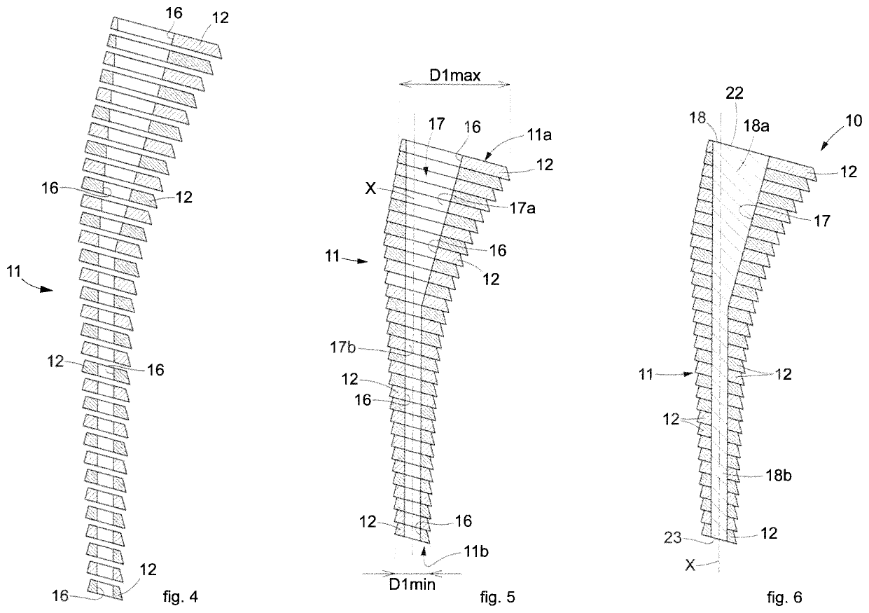

With reference to FIGS. 1, 2 and 3, the reference number 10 indicates a surgical tool according to some embodiments of the present invention.

The surgical tool 10 comprises a cutting unit 11 configured to remove and/or compact bone tissue for the preparation of a bone canal suitable to receive inside it the stem of an orthopedic prosthesis, such as for example, but not limited to, a femoral prosthesis, a prosthesis of the humerus, tibia, shoulder and others more.

We wish to clarify that in the present description and in the attached claims, with the expression “cutting”, referring to the surgical tool 10 and/or its parts, we mean the function of removing and/or compacting bone tissue.

The cutting unit 11, as a whole, is oblong in shape and is substantially tapered along its longitudinal axis X from a first end 11a (FIG. 5) to a second end 11b.

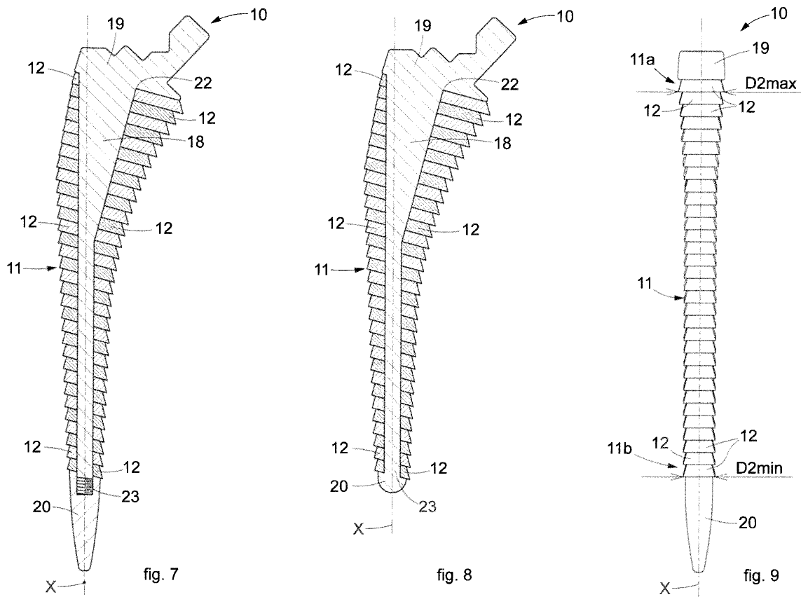

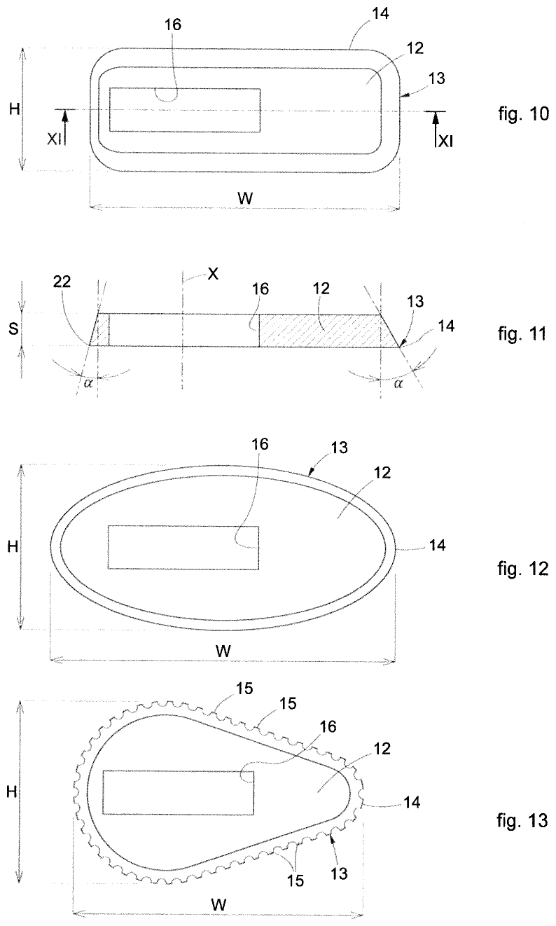

In particular, in the embodiments shown here, the cross-section of the cutting unit 11 is substantially rectangular and is defined by a first size D1 and by a second size D2 (FIG. 9), perpendicular to the first size D1 (FIG. 5). The first size D1 varies from a maximum value D1ₘₐₓ to a minimum value D1ₘᵢₙ, while the second size D2 (FIG. 9) varies from a maximum value D2ₘₐₓ to a minimum value D2ₘᵢₙ.

The trend with which the first size D1 and the second size D2 of the cutting unit 11 vary progressively along the longitudinal axis X is a function of the desired shape to be given to the bone canal that will receive the stem of the prosthesis to be implanted.

According to one aspect of the present invention, the cutting unit 11 comprises a plurality of autonomous cutting members 12 independent from each other, which are each disposed each one in contact with the adjacent one according to a determinate sequence along the common longitudinal axis X.

We wish to clarify that the longitudinal axis X as above can be either a straight line or a curved line, or a combination of rectilinear and curved segments.

Each cutting member 12 in fact substantially comprises, or consists of, a metal plate with a quadrilateral shape (FIGS. 10 and 11), preferably made of steel, or titanium, having a determinate thickness S. The cutting members 12 can even have thicknesses S different from each other. Furthermore, it is not excluded that the cutting members 12 may also have other shapes, such as, for example, an elliptical, polygonal, or round shape and others, as exemplified in FIGS. 12 and 13.

Each cutting member 12, viewed in plan (FIGS. from 10 to 13) has a determinate height H and a determinate width W, which may not coincide with the first size D1 and second size D2.

In the embodiments described here, the cutting members 12 have the height H and the width W different from each other and the sequence in which they are disposed along the longitudinal axis X is such as to give the cutting unit 11 the desired tapered profile.

Furthermore, the lateral walls of each cutting member 12 are inclined in the direction of the thickness S by an angle α with respect to the longitudinal axis X. This angle α is variable according to the considered side of the cutting member 12 and it is comprised between about 10° and about 40°.

Some embodiments of the present invention can provide that the angle α can have other values, even negative, or that it can be substantially equal to zero.

Each of the cutting members 12 also comprises at least one cutting element 13.

In accordance with the embodiment shown in FIGS. 1, 10, 11 and 12, the cutting elements 13 of each cutting member 12 can comprise one or more lateral edges 14, each of which can be sharpened, or beveled, connected or rounded, so that the surgical tool 10 can be considered a broach.

In accordance with the embodiment shown in FIGS. 2 and 13, the cutting elements 13 comprise a plurality of protrusions 15 which are made on the lateral walls of each cutting member 12, or, alternatively, in correspondence with a lateral edge 14 (FIG. 13) of the cutting member 12, so that the surgical tool 10 can be considered a rasp.

In one embodiment, not shown in the drawings but easily understood by a person of skill in the art, the cutting members 12 of the cutting unit 11 are solid, that is, without empty spaces inside them, and are mechanically connected to each other, for example by means of welding points or lines, or other types of suitable connection, for example by gluing.

In the embodiments described here, each cutting member 12 instead comprises a central cavity 16 (FIGS. from 3 to 8) which, in cooperation with the central cavities 16 of the cutting members 12, defines a central channel 17 (FIG. 5) of the cutting unit 11.

The central cavity 16 of each cutting member 12 is mating in shape with the next or previous cutting member 12 along the longitudinal axis X, in order to give the central channel 17 a desired shape.

In particular, the central channel 17 comprises a first tapered portion 17a and a second rectilinear portion 17b; although the central channel 17 can have other shapes, for example a totally tapered shape, or a totally rectilinear shape or others.

Preferably, the surgical tool 10 also comprises a central support member 18 inserted in the central channel 17 and configured to give stability and strength to the cutting unit 11.

The central support member 18 is mating in shape with the central channel 17, preferably with a minimum clearance. In particular, in the embodiments shown here, the central support member 18 comprises a first tapered portion 18a and a second rectilinear portion 18b (FIG. 6). The fact that the central channel 17 and the central support member 18 are mating in shape and that they comprise at least a first tapered portion is particularly advantageous, since it allows to verify that the cutting members 12 are disposed in the correct sequence.

The cross-section of the central cavities 16 and of the central support member 18 is, preferably but not limited to, quadrilateral, in such a way as to prevent rotation of the cutting members 12 around the longitudinal axis X (FIGS. 10, 12 and 13).

The surgical tool 10 also comprises a first element 19 associated with the cutting unit 11 and comprising a connector configured to be connected to an external tool holder.

In an embodiment not shown, the cutting member 12 at the first end 11a of the cutting unit 11 is mechanically connected, for example by welding or gluing, to the first element 19.

Instead, in the embodiments shown here that provide the presence of the central support member 18, the first element 19 is made in a single body with the latter, and it is disposed at a first end 22 thereof.

In addition, the surgical tool 10 also comprises a second element 20 connected to the central support member 18, in particular to a second end 23 thereof, opposite the first end 22. The second element 20, in cooperation with the first element 19, prevents the cutting unit 11 from sliding out of the central support member 18.

In particular, with reference to FIG. 7, the second end 23 of the central support member 18 comprises a coupling portion configured to mechanically and reversibly connect to the second element 20. This conformation is particularly advantageous since it allows to disconnect the second element 20 from the central support member 18, allowing to replace the cutting unit 11, which can therefore be of the disposable type.

Alternatively, in possible embodiments, an example of which is shown in FIG. 8, the second element 20 can be made in a single body with the central support member 18.

In other possible variants (not shown), the first element 19 can be mechanically and reversibly connected to the first end 22 of the central support member 18, for example by means of a threaded coupling.

It should be noted that even in these preferential embodiments which provide the presence of the central support member 18, the cutting members 12 can be connected mechanically, one to the adjacent one, for example by means of welding points or lines, or other types of suitable mechanical connection, for example by gluing. Alternatively, the cutting members 12 are not welded or glued to each other but they can be retained together by an external retaining mean I which can comprise, or consist of, for example, a casing of plastic material which envelops them.

The present invention also concerns a method to produce a surgical tool 10 as described here.

In particular, the method comprises, at least, the following steps:
producing, or having available, a plurality of cutting elements 12, each one distinct and separate from the other;
disposing the cutting members 12 each one in contact with the adjacent one according to a determinate sequence along a common longitudinal axis X in order to make up a cutting unit 11.

According to one embodiment, the production of the cutting members 12 provides to make a central cavity 16 in each one of them.

In these cases, the method can also comprise a step of producing, or making available, a central support member 18 mating in shape with the central cavities 16 and the step of disposing the cutting members 12 in sequence is carried out by directly inserting each cutting member 12 on the central support member 18.

Alternatively, the step of disposing the cutting members 12 in sequence can be carried out with the aid of an external template mating in shape with the central cavities 16, and in which the cutting members 12 are inserted in sequence in order to make up the cutting unit 11, which will be subsequently removed from the template and inserted on the central support member 18.

In some embodiments, the method also comprises a connection step in which the cutting members 12 disposed in sequence are mechanically connected each one with the adjacent one. The connection is preferably made by welding but other types of suitable mechanical connection, such as gluing, are not excluded.

Alternatively, the connection step can provide to envelop the cutting unit 11 in a retaining mean I, which can be a casing of plastic material configured to retain them together.

It should be noted that the connection step as above can be carried out both when the cutting members 12 are disposed in sequence with the aid of the external template as above, and also when the cutting members 12 are disposed in sequence inserted directly on the central support member 18.

The production of the plurality of cutting members 12 and/or of the central support member 18 is preferably carried out by means of the three-dimensional laser cutting of a metal plate, for example of steel or titanium, having a thickness S. This is particularly advantageous because it considerably reduces the times, and consequently the costs, required to produce the cutting unit 11, especially when compared with the times required by conventional machining with removal of chip. Furthermore, this allows to customize the profile of the cutting unit 11 resulting from the sequence of the cutting members 12, modifying their shape as required.

Alternatively, the production of the plurality of cutting members 12 and/or of the central support member 18 can be carried out by means of one of the following processes: wire-cut EDM, milling, punching or coining of a metal plate, or by means of additive manufacturing technologies such as, for example, 3D printing of metals.

In addition, the method can provide the following steps:
constraining the cutting unit 11 to the central support member 18 by means of a first element 19 disposed at a first end 22 of the central support member 18 and a second element 20 disposed at a second end 23 of the central support member 18.

It is clear that modifications and/or additions of parts or steps may be made to the surgical tool 10 and to the method as described heretofore, without departing from the field and scope of the present invention as defined by the claims.

It is also clear that, although the present invention has been described with reference to some specific examples, a person of skill in the art shall certainly be able to achieve many other equivalent forms of surgical tool 10 and/or corresponding production method, all coming within the field and scope of the present invention.

In the following claims, the sole purpose of the references in brackets is to facilitate reading and they must not be considered as restrictive factors with regard to the field of protection defined by the claims.

1. A surgical tool (10) configured to prepare a bone canal, comprising at least one cutting unit (11) configured to remove and/or compact bone tissues, wherein said cutting unit (11) comprises a plurality of autonomous cutting members (12) independent from each other and disposed each one in contact with the adjacent one according to a determinate sequence along a common longitudinal axis (X).

2. A surgical tool (10) as in claim 1, wherein it also comprises a first element (19) associated with said cutting unit (11) and configured to be mechanically connected to an external tool holder.

3. A surgical tool (10) as in claim 1, wherein each of said cutting members (12) comprises a central cavity (16) substantially coaxial to said longitudinal axis (X) and that the plurality of central cavities (16) of said cutting members (12) define a central channel (17).

4. A surgical tool (10) as in claim 3, wherein the central cavity (16) of each cutting member (12) is mating in shape with the next, or previous, cutting member (12) along the longitudinal axis (X), in order to give the central channel (17) a desired shape, the central channel (17) comprising in particular a first tapered portion (17a) and a second rectilinear portion (17b).

5. A surgical tool (10) as in claim 1, wherein the cutting unit (11), as a whole, is oblong in shape and is substantially tapered along a longitudinal axis (X) thereof, from a first end (11a) to a second end (11b).

6. A surgical tool (10) as in claim 3, wherein it also comprises a central support member (18) disposed inside said central channel (17) and configured to give structural rigidity to said cutting unit (11).

7. A surgical tool (10) as in claim 5, wherein said first element (19) is connected to a first end (22) of said central support member (18) wherein a second element (20) is connected to a second end (23) of said central support member (18), opposite said first end (22).

8. A surgical tool (10) as in claim 7, wherein at least one of either said first element (19) or said second element (20) is made in a single body with said central support member (18).

9. A surgical tool (10) as in claim 7, wherein said first element (19) comprises a connector configured to be connected to an external tool holder.

10. A surgical tool (10) as in claim 7, wherein the cutting member (12) at the first end (11a) of the cutting unit (11) is mechanically connected, by welding or gluing, to the first element (19).

11. A surgical tool (10) as in claim 7, wherein the first element (19) is made in a single body with said central support member (18), and it is disposed at a first end (22) thereof.

12. A surgical tool (10) as in claim 7, wherein said second element (20), in cooperation with the first element (19), prevents the cutting unit (11) from sliding out of the central support member (18).

13. A surgical tool (10) as in claim 7, wherein the second end (23) of the central support member (18) comprises a coupling portion configured to be mechanically and reversibly connected to the second element (20), in such a way as to allow to disconnect the second element (20) from the central support member (18), allowing to replace the cutting unit (11).

14. A surgical tool (10) as in claim 7, wherein the second element (20) is made in a single body with the central support member (18).

15. A surgical tool (10) as in claim 7, wherein the first element (19) is mechanically and reversibly connected to the first end (22) of the central support member (18), in particular by means of a threaded coupling.

16. A surgical tool (10) as in claim 4, wherein said central support member (18) is mating in shape with the central channel (17), said central support member (18) comprising a first tapered portion (18a) and a second rectilinear portion (18b).

17. A surgical tool (10) as in claim 4, wherein the cross-section of the central cavities (16) and of the central support member (18) is quadrilateral, so as to prevent the rotation of the cutting members (12) around the longitudinal axis (X).

18. A surgical tool (10) as in claim 1, wherein each of said cutting members (12) is connected mechanically, or by welding, with another adjacent one of said cutting members (12).

19. A surgical tool (10) as in claim 1, wherein each cutting member (12) in fact substantially comprises, or consists of, a metal plate having a determinate thickness (S).

20. A surgical tool (10) as in claim 19, wherein each cutting member (12) has lateral walls inclined in the direction of the thickness (S) by an angle α with respect to the longitudinal axis (X).

21. A surgical tool (10) as in claim 20, wherein said angle α is variable depending on the considered side of the cutting member (12) and it is comprised between about 10° and about 40°.

22. A surgical tool (10) as in claim 1, wherein the cutting members (12) have a quadrilateral shape, or an elliptical, polygonal, or round shape, or other.

23. A surgical tool (10) as in claim 1, wherein each cutting member (12), viewed in plan, has a determinate height (H) and a determinate width (W), wherein the cutting members (12) have a different height (H) and width (W) from each other and the sequence in which they are disposed along the longitudinal axis (X) is such as to give the cutting unit (11) a desired tapered profile.

24. A surgical tool (10) as in claim 1, wherein each of the cutting members (12) also comprises at least one cutting element (13).

25. A surgical tool (10) as in claim 24, wherein the cutting elements (13) of each cutting member (12) comprise one or more lateral edges (14).

26. A surgical tool (10) as in claim 24, wherein the cutting elements (13) comprise a plurality of protrusions (15) made on the lateral walls of each cutting member (12), or, alternatively, in correspondence with a lateral edge (14).

27. A method to produce a surgical tool (10), wherein it comprises at least the following steps:
producing, or having available, a plurality of cutting members (12), each one distinct and separate from the other; and
disposing said cutting members (12) each one in contact with the adjacent one according to a determinate sequence along a longitudinal axis (X) in order to make up a cutting unit (11).

28. A method as in claim 27, wherein in each of said cutting members (12) there is produced a central cavity (16) which is substantially coaxial to said longitudinal axis (X), so that the plurality of central cavities (16) of all said cutting members (12) defines a central channel (17), wherein it also comprises the following steps:
producing, or having available, a central support member (18) mating in shape with said central channel (17); and
inserting each of said cutting members (12) on said central support member (18) in accordance with said determinate sequence.

29. A method as in claim 27, wherein said cutting members (12) are made by means of one of the following processes: laser cutting, wire-cut EDM, milling, punching, coining of a metal plate; or by means of additive manufacturing technologies.
